(19) United States
(12) Patent Application Publication
(10) Pub. No.: US 2013/0204096 A1
(43) Pub. Date: Aug. 8, 2013

(54) Medical Device with a Deflectable Shaft Section and Tension Control

(71) Applicants: Vincent Wenchung Ku, Palo Alto, CA (US); Eric K.Y. Chan, San Carlos, CA (US)

(72) Inventors: Vincent Wenchung Ku, Palo Alto, CA (US); Eric K.Y. Chan, San Carlos, CA (US)

(73) Assignee: RUI XING LIMITED, Hong Kong (HK)

(21) Appl. No.: 13/366,034

(22) Filed: Feb. 3, 2012

(51) Int. Cl. A61B 5/00; A61B 5/04; A61B 1/00

(52) U.S. Cl. 600/301; 600/146; 600/104; 600/373

(57) ABSTRACT

A catheter comprises an elongated shaft having a proximal shaft section and a shapeable shaft section distal to the proximal section, an inner lumen extending within the proximal and shapeable shaft sections; a shaping member slidably disposed within the inner lumen and extends into the shapeable shaft section; a shape memory member adjacent to the shaping member in the inner lumen of the shapeable shaft section, wherein the shaping member and the shape memory member are coupled by a coupling coil; and a handle having an operating lever, wherein the handle receives the proximal shaft section and the shaping member for operating the shapeable shaft section.

### FIELD OF INVENTION

The present invention generally relates to medical devices with a deflectable portion, such as catheters, and, in particular to, a catheter having a steerable and deflectable distal shaft portion with tension control of the deflectable distal shaft portion.

### BACKGROUND

The heartbeat of a healthy human occurs as a result of the transfer of electrical signals within the heart. These signals are transmitted through pathways of conductive heart tissue. Unhealthy conductive heart tissue can interfere with the passage of these regular electrical signals. This interference can disturb the normal heartbeat rhythm and cause an abnormal rhythmic condition referred to as “cardiac arrhythmia.”

A widely accepted non-pharmacologic treatment for arrhythmia involves selectively destroying the unhealthy conductive heart tissue with energy such as radio frequency (“RF”) energy. In such cases, an RF ablation procedure is performed to eliminate the electrical signal irregularities by desiccating the tissue causing the arrhythmia or interfering with the transmission of such signal irregularities; the dessicated tissue is no longer electrically viable, effectively forming a barricade that prevents the conduction of electrical signal irregularities. Successful ablation of the conductive tissue at the arrhythmia initiation site usually terminates the arrhythmia or at least moderates the heart rhythm to acceptable levels. Such ablation can be performed by an electrophysiological (EP) catheter that is percutaneously introduced into the patient to perform the ablation procedure.

EP catheters are generally either tip ablation catheters or linear ablation catheters. A tip ablation catheter typically includes a longer (e.g. 4 mm to 8 mm) bullet-shaped tip ablation electrode that is made from platinum-iridium, along with shorter annular sensing electrodes (e.g. 1 mm) that are proximal to the tip. A linear ablation catheter typically includes a plurality of spaced apart electrodes, such as coil electrodes, located at the distal end of the catheter and arranged in a linear array. These electrodes are used for both sensing and energy delivery.

With respect to EP catheters, RF energy is applied through the electrode(s) to the heart tissue to produce a series of long linear lesions (e.g., via “drag and burn” methods for tip ablation catheters, or via direct placement of the distal linear array of electrodes for linear ablation catheters), that destroy the irregularly electrical-conducting tissue or interfere with the transmission of irregular signals. The cardiac electrophysiologist may choose to ablate the endocardial tissue of the heart, while the surgeon may choose to ablate the epicardial surface of the heart.

The catheters currently used for endocardial RF ablation procedures are typically flexible at the distal end, and the profile at the distal end is adjustable. Placement of the catheter distal section to achieve good tissue contact is more easily accomplished when the catheter is steerable, shapeable and deflectable.

Changing the profile of the distal shaft portion of the catheter typically involves the use of a shaping member (e.g., a wire strand) slidably disposed within an inner lumen of the catheter shaft and having its distal end attached to the distal shaft section of the catheter. The proximal end of the shaping member is attached to a lever or other element on the handle of the catheter.

The profile of the distal shaft section of the catheter shaft can be adjusted from an essentially straight configuration to a deflected or shaped configuration by applying a tensile or compressive force on the proximal portion of the shaping member. When the force is released, the distal shaft section of the catheter tends to spring back to its original or straight configuration due to the structure of the catheter shaft.

Manual maintenance of the tension or compression during the course of a medical procedure to retain the deflected or shaped distal profile of the catheter is difficult. In some existing steerable catheters, an additional knob is attached to the handle to lock the displacement of the shaping member in tension or compression to hold the distal shaft section in the desired shape. However, this is difficult and inconvenient for the operating physician.

A steerable catheter is described in U.S. Pat. No. 6,652,506 which has a locking mechanism integrated with the deflection mechanism. However, in the system described, the locking mechanism is disengaged to allow the deflection mechanism to function.

Thus, there remains a need for a steerable catheter having a deflection mechanism for better contact with the target surface and having an improved lockable steering control mechanism that can be manipulated and locked in a single-handed operation.

### SUMMARY OF INVENTION

Briefly, the present invention generally relates to a catheter comprising an elongated shaft having a proximal shaft section and a shapeable shaft section distal to the proximal section, an inner lumen extending within the proximal and shapeable shaft sections; a shaping member slidably disposed within the inner lumen and extends into the shapeable shaft section; a shape memory member adjacent to the shaping member in the inner lumen of the shapeable shaft section, wherein the shaping member and the shape memory member are coupled by a coupling coil; and a handle having an operating lever, wherein the handle receives the proximal shaft section and the shaping member for operating the shapeable shaft section.

The present invention is also generally directed to a medical device such as an electrophysiology-type catheter that has an operating handle and a shapeable or deflectable distal shaft section to facilitate placement of the catheter to a desired location within a patient's body. The catheter has a self-locking deflection system which facilitates placing and holding the distal shaft section of the catheter in a desired shape or state of deflection. The steering system can be manipulated by an operator with a single hand to establish a desired distal shaft section shape and lock the distal shaft section in the desired shape with a single operation.

These and other features of the invention will become more apparent from the following detailed description and the accompanying drawings.

### DESCRIPTION OF THE DRAWINGS

The foregoing and other objects, aspects, and advantages of the invention can be better understood from the following detailed description of the preferred embodiment of the invention when taken in conjunction with the accompanying drawings in which:

FIG. 1 illustrates an elevational view of a catheter of the present invention having an elongated catheter shaft secured to a handle which has a movable lever for deflecting or shaping the distal shaft portion of the catheter.

FIGS. 1A-1B illustrate enlarged elevational views of the shapeable shaft section of a catheter of the present invention in curved and extended positions, respectively.

FIG. 2 illustrates an enlarged longitudinal cross-sectional view of the distal section of a catheter of the present invention.

FIG. 3 illustrates a transverse cross-sectional view of the distal portion of a catheter of the present invention.

FIG. 4 illustrates an enlarged longitudinal cross-sectional view of a catheter of the present invention.

FIG. 5 illustrates an elevational view of the distal portion of the deflection mechanism of a catheter of the present invention, in which the polymeric tubing is not present.

FIG. 6 illustrates an elevational view of a proximal part of the deflection mechanism of a catheter of the present invention, in which the electrodes and outer sheaths are removed and the polymeric tubing of the elongated shaft is shown.

FIG. 7 illustrates an exploded view of the side wall of the self-locking deflection system in FIG. 1 to illustrate details of a rotor secured thereto.

FIG. 8 illustrates a longitudinal cross-sectional view of the handle of a catheter of the present invention.

FIG. 9 illustrates an enlarged longitudinal cross-sectional view of the distal portion of the handle of a catheter of the present invention.

FIG. 10 illustrates a transverse cross-sectional view of the handle of a catheter of the present invention.

FIG. 11 illustrates a transverse cross-sectional view of the handle of a catheter of the present invention.

FIG. 12 illustrates a transverse cross-sectional view of the handle of a catheter of the present invention.

FIG. 13 illustrates a longitudinal cross-sectional view of the handle of a catheter of the present invention.

FIG. 14 illustrates an enlarged longitudinal cross-sectional view of the tubular support member for the shaping member of a catheter of the present invention.

FIGS. 15 and 16 illustrate longitudinal transverse cross-sectional views of a handle of a catheter the present invention with the operating lever in a proximal position for deflecting the distal shaft section of the catheter.

FIG. 17 illustrates a longitudinal cross-sectional view of the distal shaft section of a catheter of the present invention which shows details of the shaping member secured within the distal shaft section.

FIG. 18 illustrates a transverse cross-sectional view of the handle of a catheter of the present invention having an irrigation channel inside the handle.

### DETAILED DESCRIPTION OF THE PREFERRED EMBODIMENTS

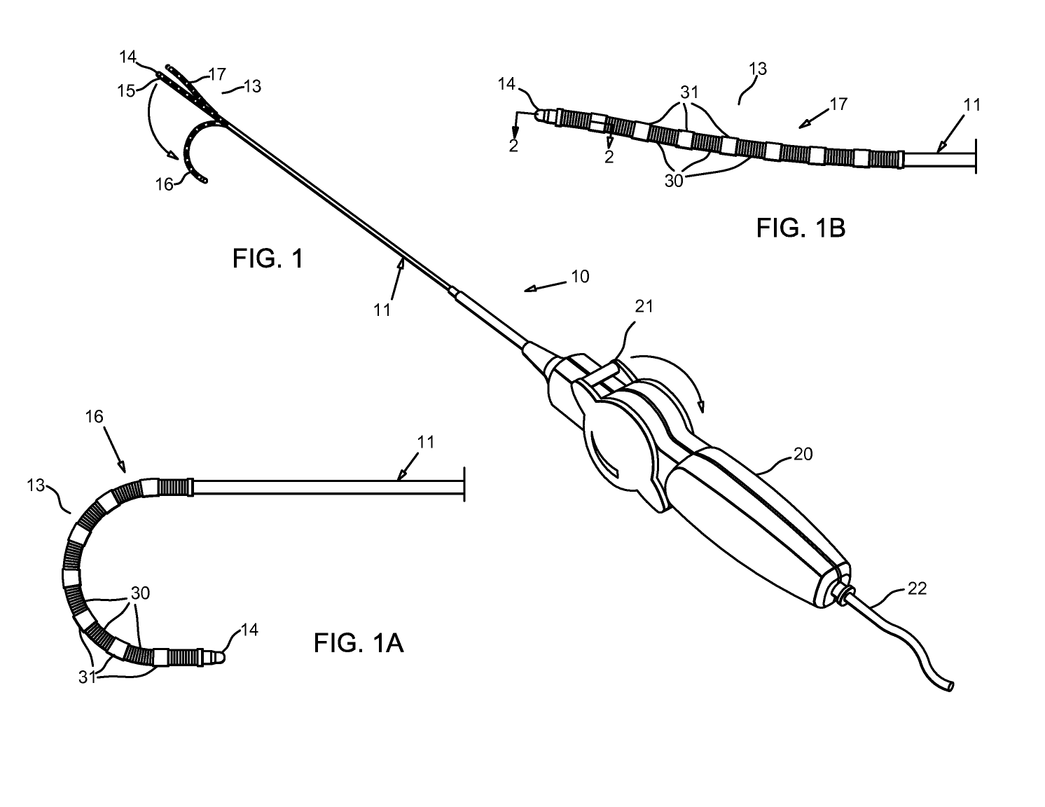

FIG. 1 illustrates an elevational view of a catheter of the present invention having an elongated catheter shaft secured to a handle which has a movable lever for deflecting or shaping the distal shaft portion of the catheter. An elongated catheter device 10 (e.g., an electrophysiology catheter) has an elongated shaft 11, a proximal shaft section 12 and a shapeable shaft section 13 distal to the proximal shaft section 12 with a non-traumatic distal end or tip 14. FIG. 1 shows the shapeable shaft section 13 in various positions, including a neutral position 15, a curved position 16, and a hyper-extended position 17. Generally, the catheter 10 has a handle 20, located proximally from the elongated shaft 11. The elongated shaft 11 has a plurality of electrodes 30 on the deflectable or shapeable distal shaft section 13. The proximal shaft portion 12 is secured to the distal end of handle 20. The operating handle 20 has a self-locking steering control or deflection system that is operated by a lever 21 for deflecting or otherwise shaping the distal shaft section 13. The lever 21 can be a wheel or dial, where a user can turn the wheel or dial to set the amount of deflection of the shapeable distal shaft section 13. The operating handle 20 also provides a power source connection 22 for supplying power to the electrodes 30.

In other embodiments of the invention, the distal shaft portion 13 of the elongated catheter device 10 can also have temperature sensing elements, such as thermocouples, underneath the electrodes 30. The temperature sensing elements or thermocouples are preferably spaced from the electrode so as to provide a response time (tᵣ), which is approximately 3 time constants (3τ) and does not exceed 4 seconds, i.e. tᵣ≅=3τ≦4 sec.

In addition, a pressure sensor can be disposed on the non-traumatic distal end 14, i.e., the distal tip of the shapeable shaft section 13. Preferably, there is a pressure sensor disposed on each of the four quadrants of the non-traumatic distal end 14. The wiring for the electrodes 30, thermocouples, and/or pressure sensors can be bundled together; the bundled wiring can be routed from the handle 20, through the inner lumen of the elongated shaft 11, to the respective electrodes, thermocouples, and/or pressure sensors.

FIGS. 1A and 1B are enlarged perspectives illustrating the shapeable shaft section 13 in the curved position 16 and hyper-extended position 17, respectively. The catheter device 10 has multiple, preferably coiled electrodes 30 between multiple polymer laminations 31 which overlap and fix the ends of the electrodes 30. The polymer laminations 31 may be composed of tetrafluoroethylene, hexafluoropropylene and vinylidene fluoride (“THV”) or another similar insulating polymer material. The atraumatic distal tip 14 may be composed of a gold solder or platinum alloy. The atraumatic distal tip 14 is configured to allow for the advancement of the catheter through body tissue and lumens with minimal harm to tissue the tip contacts.

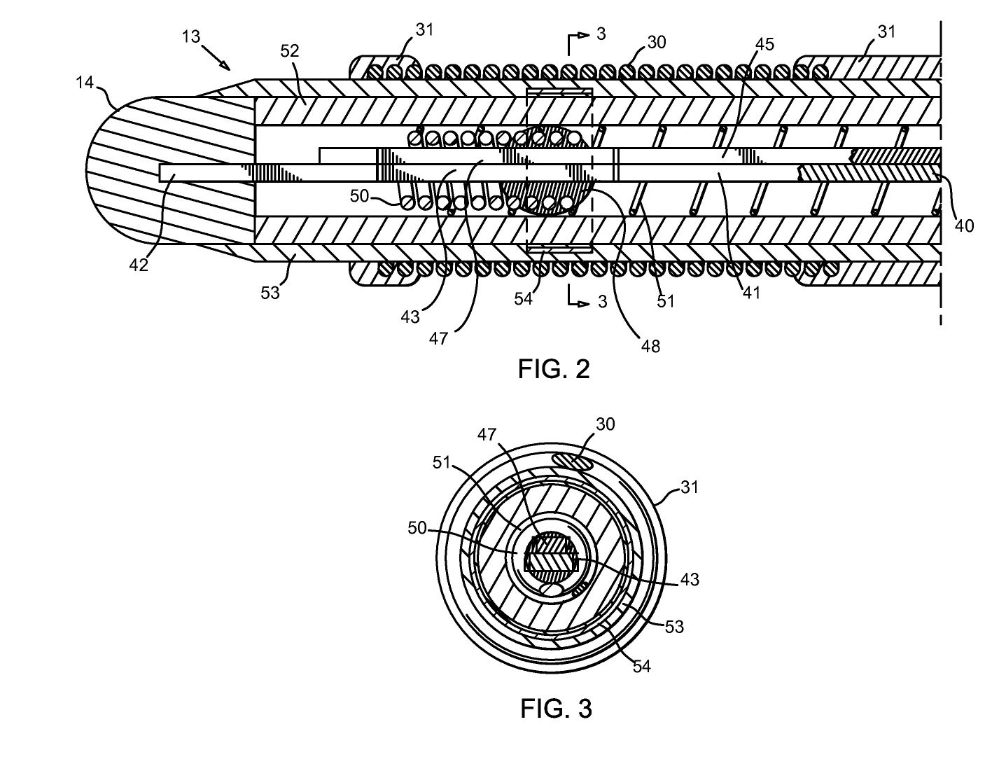

FIG. 2 illustrates an enlarged longitudinal cross-sectional view of the distal section of a catheter of the present invention, along line 3-3. In particular, the shapeable shaft section 13 of the elongated shaft 11 of a deflectable catheter 10 is shown. A shaping member 40 can have a flattened distal portion 41 that extends axially within the shapeable shaft section 13. The shaping member 40 is preferably a wire composed of stainless steel. The shaping member 40 of this embodiment can be controlled by the lever 21 on the operating handle 20 shown in FIG. 1. The shaping member's distal end 42 is affixed to the atraumatic distal tip 14.

The shape memory ribbon 45 can extend axially in the shapeable shaft section 13 and is located adjacent to the shaping member's flattened distal portion 41. Preferably, the shape memory ribbon 45 is a ribbon formed of a stress-induced martensite (SIM) NiTi alloy. Other shape memory materials may also suffice as well. Note that a shape memory ribbon can also be referred to as a shape memory member.

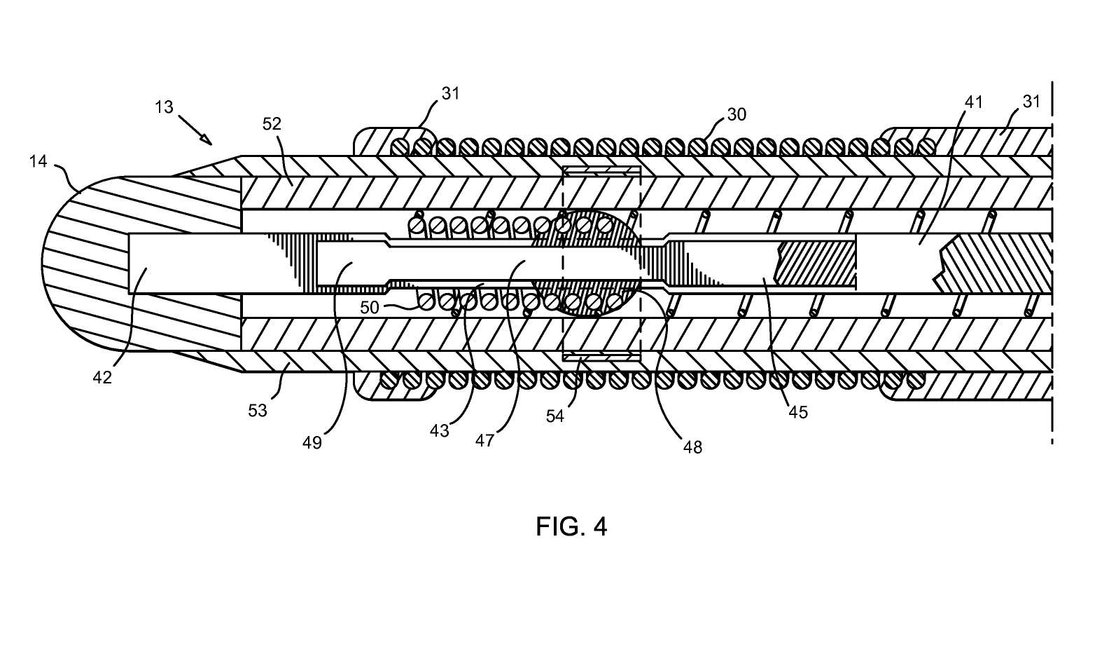

FIGS. 2-5 illustrate the manner by which the shaping member's flattened distal portion 41 and the shape memory ribbon 45 are affixed to one another in the shapeable shaft section 13. Referring to FIG. 4, the shaping member's flattened distal portion 41 has a notched section 43 proximal to and near the shaping member's distal end 42. The shape memory ribbon 45 has a corresponding notched distal section 47 proximal to and near the shape memory ribbon's distal end 49. The notched sections 43 and 47 are affixed to one another by a distal joint coil 50. Generally, a coupling coil can be used to affix the notched sections 43 and 47, such that some bowing can occur when the shaping member 40 is pulled or pushed. However, the shaping member 40 and the shape memory ribbon 45 are prevented from completely bowing apart by the coupling coil. A specific embodiment of a coupling coil is the distal joint coil 50.

The distal joint coil 50 may be composed of stainless steel. Thus the distal joint coil 50 surrounding the shaping member notched distal section 43 and the shape memory ribbon notched distal section 47 forms a mechanically secure joint. Also a body of solder 48, shown in FIGS. 2 and 4, fixes the distal joint coil 50 to the shaping member notched section 43 and the shape memory ribbon notched section 47. Those skilled in the art may choose to secure the shaping member's flattened distal portion 41 and the shape memory ribbon 45 in an alternative manner as well.

While not illustrated in the drawings, the shaping member's flattened distal portion 41 preferably has a polytetrafluoroethylene (PTFE) coating, or is surrounded by PTFE tubing. The coating or tubing can act as a lubricant so that the shaping member's flatten section 41, helical confining wire 51, and shape memory ribbon 45 can slide with respect to one another, while still occupying a confined space within helical confining wire 51.

FIGS. 2-6 show the helical confining wire 51 extending about the shaping member's flattened distal portion 41 and the shape memory ribbon 45. The helical confining wire 51 is wrapped around the shaping member 40 and the shape memory ribbon 45 to control the spacing between these two members when the shapeable shaft section 13 is deflected, hyper-extended, or otherwise shaped. Essentially the helical confining wire 51 prevents the shaping member's flattened distal portion 41 and the shape memory ribbon 45 from completely bowing apart when the shaping member 40 is pulled or pushed. This is to ensure improved tissue contact force is applied by the distal electrodes near the distal tip 14.

The shaping member 40 and the shape memory ribbon 45 can be spaced apart along the longitude of the shapeable shaft section 13 a predefined distance away from each other; this predefined distance can be set based on an amount of deflection required. The helical confining wire 51 restricts the maximum distance between the shaping member 40 and the shape memory ribbon 45 to the predefined distance. Thereby, the shape memory ribbon 45 provides a spring back force near the distal tip 14 of the shapeable shaft section 13 that is opposite to the movement and/or curvature of the shapeable shaft section 13. For instance, when the shapeable shaft section 13 is deflected, the shape memory ribbon 45 can slightly curve the shapeable shaft section 13 near the distal tip 14 in opposition to the movement and/or curvature of the shapeable shaft section 13. In such a manner, improved tissue contact can be provided at the distal electrodes of the shapeable shaft section 13 near the distal tip 14.

FIGS. 2-4 illustrate a braiding layer 52 that surrounds the helical confining wire 51, further confining the movement of the shaping member's flattened distal portion 41 and shape memory ribbon 45. Alternative ways to control the space between the shaping member's flattened distal portion 41 and the shape memory ribbon 45 include a wire mesh or other structures.

The helical confining wire 51 and braiding layer 52 can be located within a polymeric tube 53; the latter being preferably formed of vinylidene fluoride (THV) or equivalent. The coiled electrodes 30 are located about the polymeric tube 53. FIGS. 2-4 show the most distal of the multiple coiled electrodes 30. Between the coiled electrodes 30, also about the polymeric tube 53, are circumferential bands of polymer electrode end fixing lamination 31. The lamination 31 may be formed of tetrafluoroethylene, hexafluoropropylene and vinylidene fluoride (THV). Located beneath the coiled wire electrodes 30 are thermocouple bands 54 capable of accurate temperature measurements. The bands 54 are preferably spaced from the coiled wire electrodes 30 to provide a 3 tau response time of less than four seconds. The bands 54 may be spaced from coiled electrodes 30 by a suitable polymeric material such as an extruded viscoelastic polymer selected from the group of polyethylene and polypropylene. FIGS. 2-4 only depict the most distal of the multiple thermocouple bands 54. The thermocouple bands 54 are preferably formed of gold.

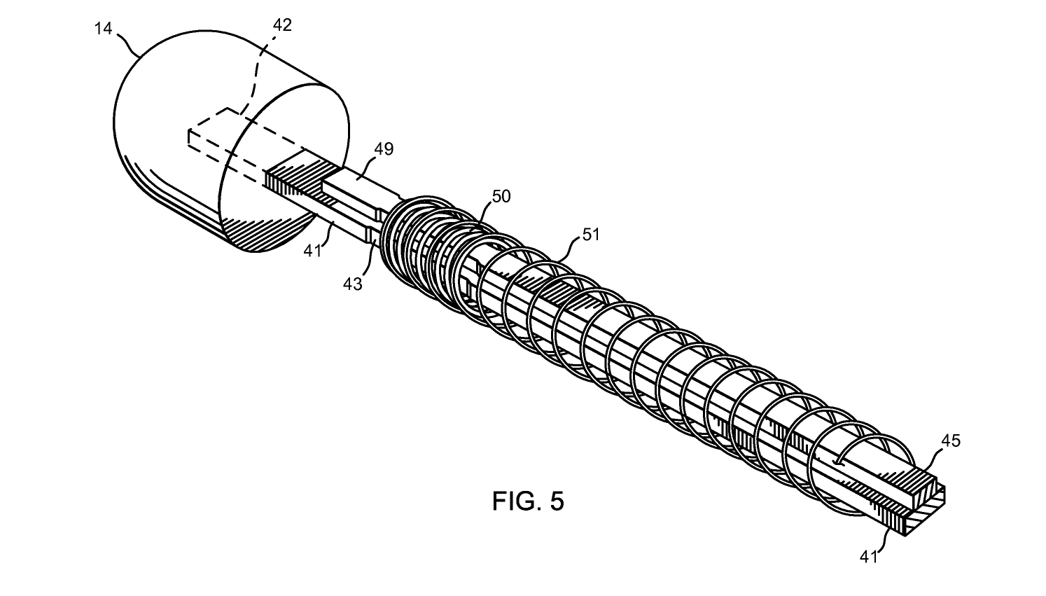

FIG. 5 illustrates an elevational view of the distal portion of the deflection mechanism of a catheter of the present invention, in which the polymeric tubing is not present. The distal shaft section 13 is shown after removing the braiding layer 52, polymeric tubing 53 and coiled electrodes 30 from view. The shaping member's flattened distal portion 41 and the shape memory ribbon 45 are bound together by a helical confining wire 51. A mechanically fixed joint formed by the distal joint coil 50 binding the distal shaping member notched section 43 and the distal shape memory ribbon notched portion is also illustrated.

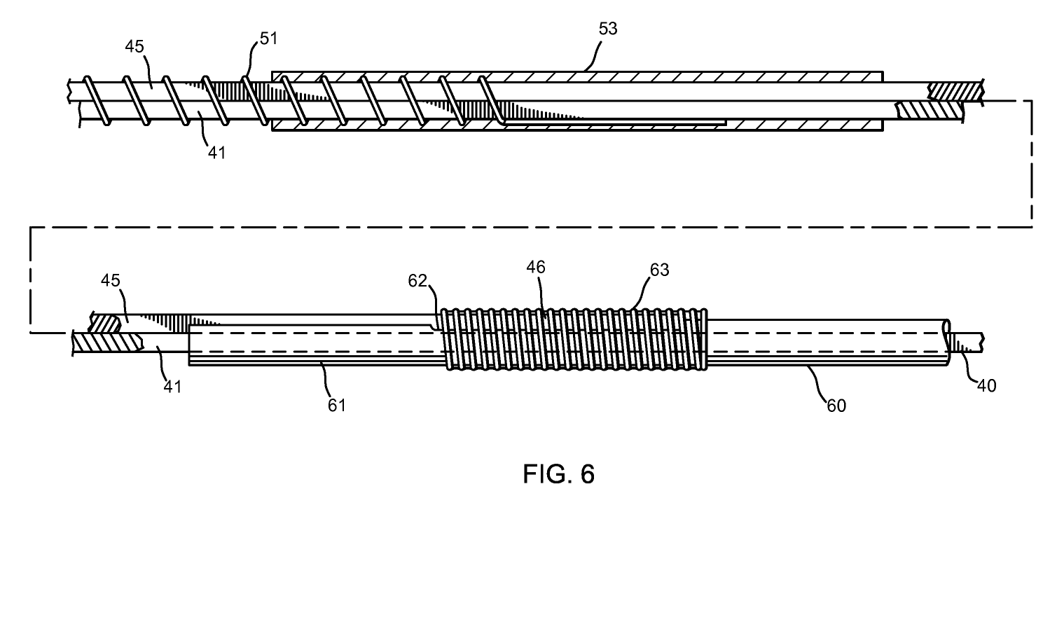

FIG. 6 illustrates an elevational view of a proximal part of the deflection mechanism of a catheter of the present invention, in which the electrodes and outer sheaths are removed and the polymeric tubing of the elongated shaft is shown. The polymeric tube 53 is cut-away to allow for viewing of the joint formed by the shape memory ribbon proximal end 46 and the hypotube 60. The shape memory ribbon's proximal end 46 and a recessed section 62 of the hypotube distal portion 61 are configured to form a splice joint. While not illustrated in FIG. 6, the articulating surfaces of the shape memory ribbon's proximal end 46 and the recessed hypotube section 62 may be additionally secured with a metal adhesive. The shape memory ribbon proximal end 46 may also be secured with adhesive to the interior of the proximal end of the polymeric tube 53. A hypotube joint coil 63 circumferentially surrounds and further secures the joint formed by shape memory ribbon's proximal end 46 and the hypotube distal portion 61. The proximal portion of the shaping member 40 extends proximally within the hypotube 60 toward the handle 20. As discussed previously, the handle 20 controls the pushing and pulling of the shaping member 40. FIG. 6 represents just one illustration of how the proximal end of the shape memory ribbon 46 is secured. Those skilled in the art may choose to secure the proximal end of the shape memory ribbon 46 in a different manner.

In one presently preferred embodiment, an EP catheter of the present invention as described above is inserted through an opening in a patient's femoral vein and advanced within the patient's venous system until the shapeable shaft section is within the patient's vena cava. The lever on the operating handle is pulled proximally to put the shapeable shaft section in a circular shape and placed on target tissue. High frequency electrical current is supplied to a plurality of the electrodes on the shapeable shaft section 13 so as to form a continuous lesion in the endocardial surface of the patient's heart in order to terminate arrhythmia in the patient's heart.

The typical dimensions of an EP catheter of the present invention such as shown in the drawings are as follows:

| Catheter Component | Dimension |
|---|---|
| Distal Tip Outer Diameter | 0.052″ ± 0.005″ (4 F, 1.14 mm) |
| Electrode Region Outer Diameter | 0.074″ ± 0.005″ (5.6 F, 1.9 mm) |
| Proximal Shaft Outer Diameter | 0.071″ ± 0.005″ (5.4F, 1.8 mm) |
| Electrode Length | 4 mm ± 0.05 mm |
| Electrode Spacing | 0.6 mm ± 0.5 mm |
| Thermocouple Band Length | 1.75 mm ± 0.25 mm |
| Electrode Region Length | 40 mm ± 5 mm |
| Working Length of Catheter | 110 cm ± 3 cm |
| Proximal Adapter Length | 22 cm ± 3 cm |
| Distal Tip Length | 2 mm ± 1 mm |
| Curve Dia. of Distal End | 1.5 cm to 2.5 cm |
| Extension of Distal End | 0.5 cm minimum |

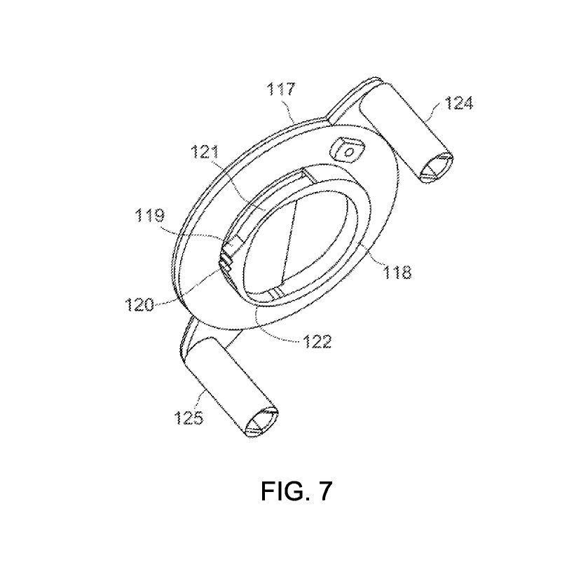

FIG. 7 illustrates an exploded view of the side wall of the self-locking deflection system shown in FIG. 1. A side wall 117 of a self-locking deflection system for the catheter device has a rotor 118 that has an engagement member 119. The engagement member 119 has teeth 120 and is secured to the rotor 118 by a pair of spring members 121 and 122. Operating levers 124 and 125 are attached to the side wall 117 for rotating the rotor 118. Either or both of the operating levers 124 and 125 can be used to rotate the rotor 118. For purposes of clarity, the detailed description may refer to using a single operating lever, however, it is understood that either or both of the operating levers can be used for rotating the rotor 118.

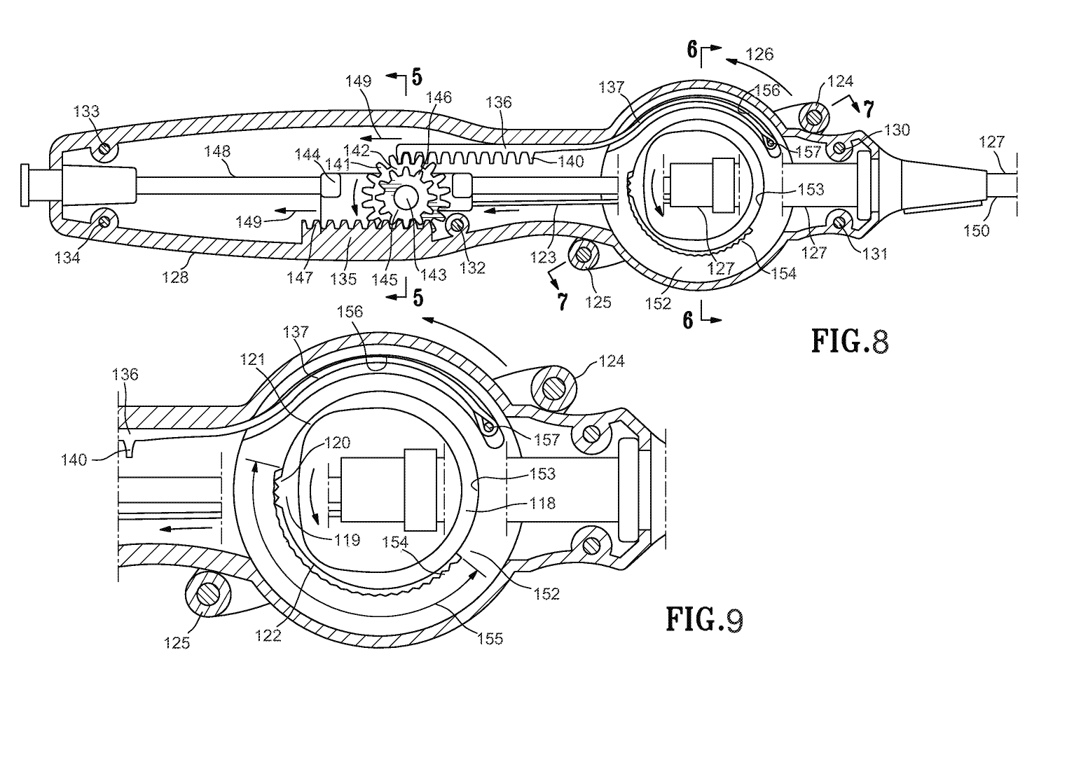

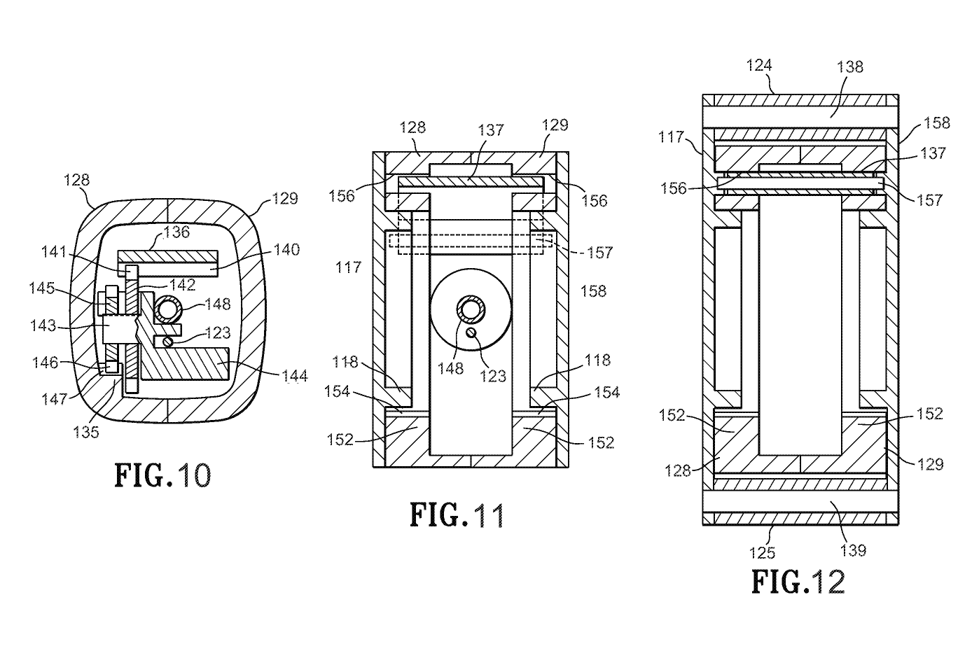

FIG. 8 illustrates a longitudinal cross-sectional view of the handle of a catheter of the present invention. FIG. 9 illustrates an enlarged longitudinal cross-sectional view of the distal portion of the handle and FIGS. 10, 11, 12 illustrate transverse cross-sectional views of the handle shown in FIG. 8 taken along lines 5-5, 6-6, and 7-7, respectively.

As shown in FIGS. 8, 9 10, 11, and 12, a deflecting or shaping member 123 of the catheter device extends from the elongated shaft of the catheter device into the handle. Therein, the shaping member 123 is secured within the handle of the catheter device to the self-locking deflection system. To operate the deflecting or shaping member 123, the self-locking deflection system is operated by operating levers 124 and 125 which move in the direction shown by arrow 126. Tensile or compressive forces applied to the proximal portion of shaping member 123 by the operating levers 124 and 125 causes the deflection and/or shaping of the distal shaft portion of the catheter. The shaping member 123 is preferably slidably disposed within tubular support member 127 which provides columnar support thereto.

The handle for a catheter of the present invention can have two half shells, a left side half shell and a right side half shell. FIGS. 8 and 9 illustrate the interior of the left side half shell. The left side half shell is secured to the right side half shell by a plurality of pins 130-134. Minor details of the interior have been omitted from FIGS. 8-9 for purposes of clarity. The lower base of left side half shell has a fixed rack 135 and the upper portion of the left side half shell is provided with a flexible rack 136. The flexible rack 136 has a flexible belt frame 137 which is secured to a pin 157 that passes through the self-locking deflection system. The flexible rack 136 can be made of nylon plastic, metal alloy, or other material. The teeth 140 of the flexible rack 136 engage the teeth 141 of the large gear 142 which is rotatably secured to a post 143. The post 143 is fixed to a slider 144. The small gear 145 is rotatably secured to the post 143 and the teeth 146 of the small gear 145 engage the teeth 147 of the fixed rack 135.

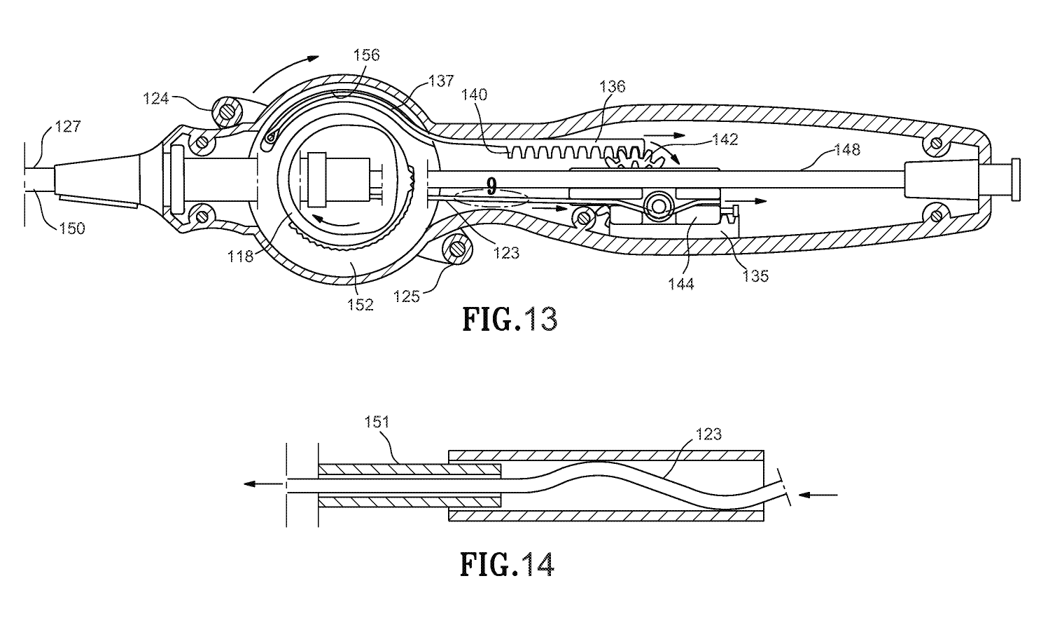

Movement of the operating lever 124 (and/or operating lever 123) in the direction of arrow 126 pushes on the flexible lead 137 of the flexible rack 136, moving it toward the proximal end of handle. Proximal movement of the flexible rack 136 rotates the larger gear 142 fixed to the post 143 which in turn rotates the small gear 145, which is likewise fixed to the post 143. The small gear 145 rotates along the fixed rack 135. The slider 144 is slidably mounted to a tubular member 148 which extends through the handle. When the slider 144 is urged proximally as shown by arrow 149 and rides along tubular member 148, the proximal end of shaping member 123 which is fixed to the slider 144 (as shown in FIGS. 8 and 13) is therefore pulled proximally within tubular support member 127.

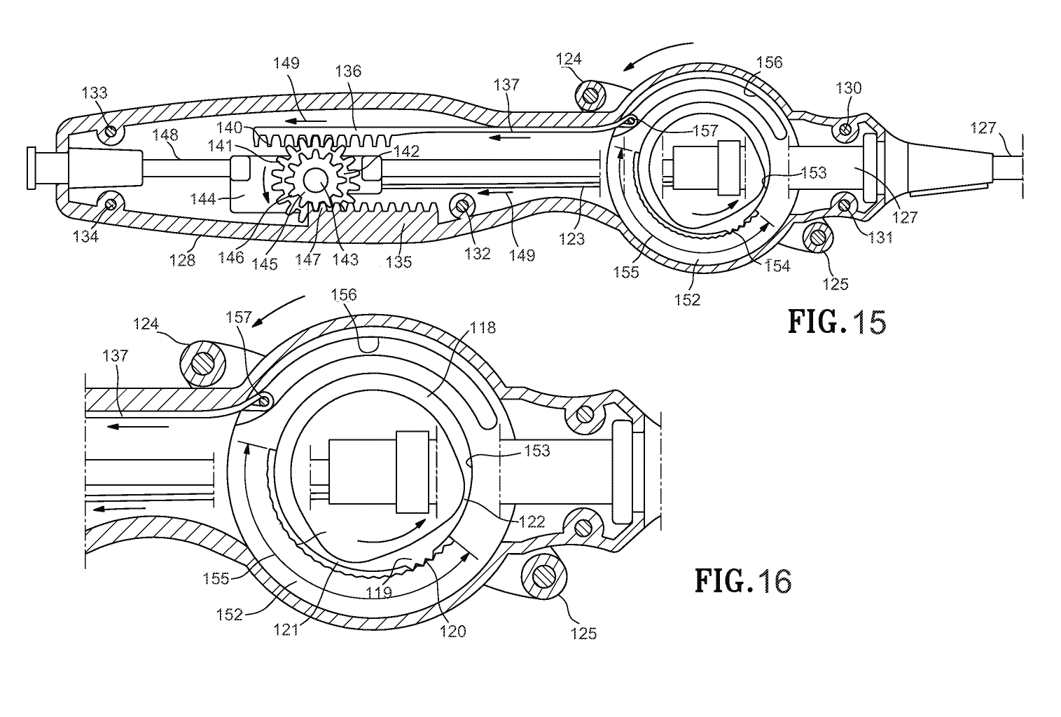

As shown in FIGS. 8, 9, 13 and 16, the self-locking deflection system includes a journal 152 within the housing 20 which is secured to a wall of the self-locking deflection system. The journal 152 has an inner surface 153 which is provided with a plurality of teeth 154 which extend along a length 155 of the inner surface 153. The teeth 154 increase in height along the length 155. The rotor 118 (shown in FIG. 7) slides into the interior of journal 152 and is configured to be slightly larger than the interior of the journal, so that the engagement member 119 and the teeth 120 thereon are pressed against the teeth 154 along the length 155 of the_inner surface 153. The springs 121 and 122 of rotor 118 apply constant pressure on the engagement member 119 so that the teeth 120 thereon are always engaging the teeth 154 on the inner surface 153 as the engagement member moves along the length 155. Thus, as the operating lever 124 is moved by the operator from the forward position as shown in FIGS. 1, 8, 13 and 16 to a proximal position as shown in FIGS. 15 and 16, the operator senses greater resistance to movement because of the gradually increasing height of the teeth 154. The increased resistance gives the operator an indication of the shape of the distal shaft section 13 of catheter 10 in addition to any indicia that may be provided on the handle or the self-locking deflection system. This increased resistance to movement also ensures that the shaped distal shaft section 13 remains in the desired position when the operator's pressure against the operating lever 124 is removed due to the pressure against the engagement member by springs 121 and 122.

While the self-locking deflection system is described utilizing an engagement member 119 with teeth 120 which engages the teeth 154 on the inner surface 153 of journal 152, a variety of biased frictional engagements may be employed. For example, similar results may be obtained with the inner surface 153 of journal 152 having increase coarseness, e.g. sandpaper, or stickiness along the length 155.

Moreover, engagement member 119 may be biased by hydraulic pressure, pneumatic pressure, compressible materials, elastic materials, magnetic forces, or by any equivalent means instead of spring members 121 and 122.

Referring to FIGS. 8, 9, 15, and 16, flexible lead 137 of flexible rack 136 is slidably disposed within the groove or raceway 156 formed in the journal 152 and the distal end of flexible lead 137 is secured to a pin 157 that is secured to the rotating side wall 117 and the other accompanying rotating wall. Movement of the operating lever 124 moves the flexible lead 137 and thus flexible rack 136. The teeth 140 of the flexible rack 136 engage the teeth 141 of large gear 142, thereby driving the slider 144 proximally along the tubular member 148. The proximal end of shaping member 123 is secured to the slider 144 so that proximal movement of the slider deflects or otherwise shapes the distal shaft section 13.

Usage of the handle 20 and movement of the operating levers 124 or 125 by an operator can be accomplished with a single hand. For example, while the operator is holding the handle in his or her hand, the lever 124 from a forward position to a proximal position with his or her thumb until the desired deflection or shaping of the distal shaft section 13 of the catheter shaft 110 is obtained. The operator may release his or her thumb from the operating lever 124, thereby holding deflected or shaped configuration of the distal shaft section

Referring to FIGS. 13 and 14, the proximal end 150 of tubular support member 127 is secured to a smaller diameter tubular member 151 which extends within the catheter shaft. When the operator pushes lever 124 in the reverse direct, the shaping member 123 is pushed along the inner wall and transits into the smaller tubular member 151, resulting in a reversing deflection of the distal shaft section 13.

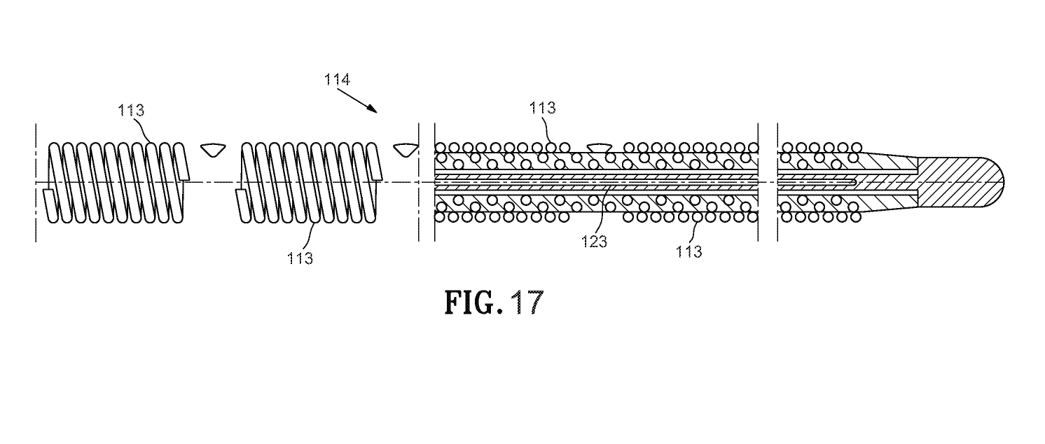

FIG. 17 illustrates the deflection shaping member 123 secured to the distal shaft section of the catheter. Generally, a plurality of electrodes 113 is disposed on an exterior 114 of the deflectable or shapeable distal shaft section. The other layers of the distal shaft section are not shown for purposes of clarity.

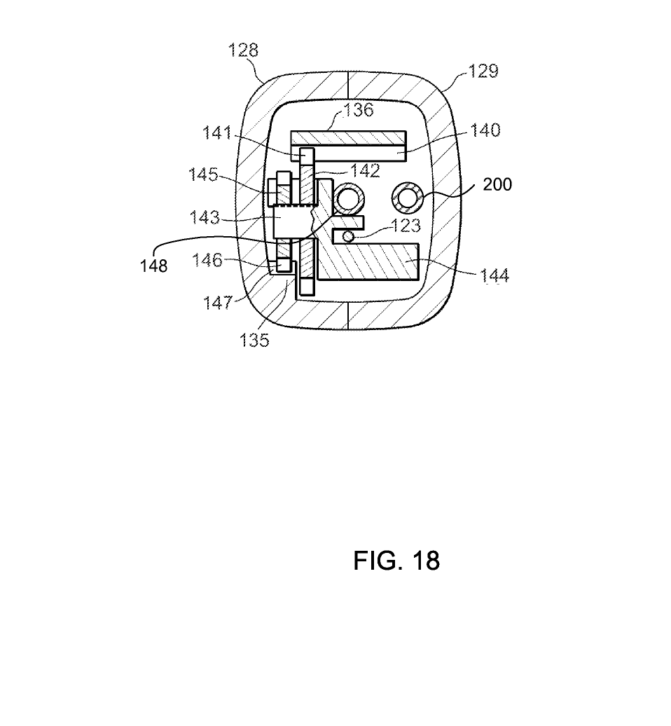

FIG. 18 illustrates a transverse cross-sectional view of a handle of the present invention, taken along the lines 5-5, having an irrigation channel in the interior of the handle. The electrophysiology catheter can include an irrigation system, where the irrigation system provides liquid to the exterior of the shapeable shaft section 13 to irrigate the electrode(s) of the electrophysiology catheter. The liquid can be saline or other solution for medical use. The distal shaft section 13 is provided with outlet openings adjacent to the electrode(s) of the catheter. The fluid can be pumped through the handle via an irrigation channel 200 to the inner lumen of the elongated shaft. Upon reaching the distal shaft section 13, the fluid can flow from the outlet openings over the exterior of the shapeable shaft section 13 where the electrodes are disposed. Thus, the irrigation system can be useful for controlling the degree of thermal effect surrounding the intended treatment site.

### An Embodiment of the Present Invention

A presently preferred catheter having features of the invention includes an elongated shaft having a proximal and distal ends with a shapeable or deflectable distal shaft section having at least one exposed electrode to perform an electrophysiology function such as sensing, ablating or both. The shapeable shaft section better maintains a profile that allows for contacting the heart surface at the time of ablation.

The elongated shaft of the catheter has at least one inner lumen extending from the proximal end of the shaft to the distal shaft section and a shaping member having proximal and distal ends extending within the inner lumen. The distal end of the shaping member is secured to the distal shaft section so that when a tensile or compressive force is applied to the shaping member, the distal shaft section will be provided with a desired shape or deflection.

The shapeable shaft section of a catheter having electrodes is placed inside the chamber of a patient's atrium, preferably in a curved shape, advanced within the patient's heart chamber to engage target tissue. The curved shapeable shaft section of the catheter is then extended into a more linear shape. As the distal catheter section is extended, some of the electrodes make tissue contact sooner than others. The lengths of the shapeable shaft portion which do not contact tissue encounter less resistance and will uncurl or straighten out more that those lengths which engage tissue and encounter a greater resistance. As a result, the shapeable shaft section of the catheter, to a certain extent, takes the shape of the irregular surface terrain of the patient's atrium. This allows for better contact facilitating a better ablated linear lesion over an uneven surface with fewer ablation procedures.

An elongated medical device embodying features of the invention includes a deflection mechanism that has an elongated shaping member such as a wire that extends through the interior thereof with a flattened distal portion. The distal end of the flattened distal portion is secured, preferably to the distal non-traumatic tip of the medical device. A shape memory member such as a ribbon or strip, preferably a stress induced martensite (SIM) NiTi alloy such as NITINOL, extends adjacent to and co-linear with at least part of the flattened distal portion of the elongated shaping member.

One or more confining members such as wires or strands extend about the shape memory member and the flattened distal portion of the elongated shaping member to control the spacing between these two members when the shapeable distal section of the device is deflected or otherwise shaped. The one or more confining members may be in the form of a helical coiled wire, a mesh member or be of a braided construction.

The elongated shaft of the medical device preferably has a stainless steel hypotube with an inner lumen that slidably receives at least part of the elongated shaping member and a proximal portion which extends into the handle of the device. The hypotube extends distally to the shapeable shaft section preferably to a location proximal to the flattened distal portion of the elongated shaping member. A polymeric tube is preferably secured to the distal end of the hypotube and extends therefrom for stress relief and kink resistance. The proximal portion of the shape memory member is secured, preferably by adhesive between the exterior of the distal end of the hypotube and the interior of the proximal end of the polymeric tube and a locking key feature. A coil or wound wire is placed over the locking key feature, fixed by solder, to further enhance the coupling joint. The polymeric tube may be formed of vinylidene fluoride or polyimide.

The distal end of the shape memory member is secured to the distal end of the flattened portion of the elongated shaping member. Preferably, the distal ends of the shape memory member and the flattened portion of the elongated shaping member have notches so that a wire or ribbon made of suitable material such as stainless steel can be wrapped around both members within the notches thereof to mechanically secure them together. A body of solder may be formed about the notches and wound wire or ribbon to further secure the members together. The distal ends of the one or more wires or strands of the confining member may also be secured within the notches by the solder. NiTi alloys are difficult to solder so the mechanical connection of the wrapped wire and the solder secures the distal end of the NiTi shape memory member to the flattened stainless steel shaping member (e.g., a wire).

The elongated shaping member and hypotube extend proximally to an operating handle. The handle is provided with a mechanism that pulls and pushes the shaping member which in turn shapes or deflects the shapeable shaft section of the elongated device. The proximal end or portion of the shaping member is secured to a force applying slider or collar that is preferably configured to follow a desired or predefined path within the catheter handle.

The handle of the steerable device receives the proximal end of the elongated shaft and has a self-locking deflection system to both move the distal shaft section into a desired configuration and hold or lock the distal shaft section in the desired configuration. The self-locking deflection system includes a first friction engaging surface with a first friction characteristic such as plurality of protuberances or teeth and a second friction engaging surface with variable friction characteristics along a length thereof such as a plurality of protuberances or teeth with increased protuberance or tooth height along a length of motion to increase the frictional resistance felt by the operator. The handle has a shaping operating member such as a lever which is directly or indirectly connected to the shaping member and to one of the first or second friction engaging surfaces. The self-locking deflection system has a movable member with one of the friction surfaces and one or more biasing elements which urge the movable member having a friction engaging surface toward the other of the friction engaging surfaces. The biasing element(s), such as springs, are configured so the friction engaging surface on the movable member is always in contact with the other friction engaging surface, even as the movable member is moving so as to control the shape or deflection of the distal shaft section.

The self-locking deflection system is operated by an operating member or lever which is movably mounted on the handle housing and which is directly or indirectly secured to the slider or collar mounted to a proximal end of the shaping member, and which is secured to a rotating or moving member having one of the friction engaging surfaces with a second set of a plurality of protuberances.

Preferably, the first and second friction engaging surfaces have undulating surfaces with a plurality of peaks and valleys. With the first of the friction engaging surfaces, the peaks of the undulating surface are about the same height. With the second friction engaging surface, the peaks of the undulating surface have increasing heights along a length of the undulating surface to provide an increase in the resistance between the first and second friction surface as the moving member moves in the moving direction. At least one of the peaks of the first friction engaging surface is always in a valley of the second friction engaging surface and the biasing element(s) ensure contact between the first and second friction engaging surfaces.

With the present invention, there is preferably no disengagement of the friction engaging surfaces, so the distal shaft section is automatically locked in the desired position. The increased resistance felt by the operator provides that operator with an indication of the shape of the distal shaft section.

Preferably, an electrophysiology catheter of the present invention includes an irrigation system, where the irrigation system provides liquid to the exterior of the shapeable shaft section for irrigating the electrode(s) of the electrophysiology catheter.

While the medical device described herein is a EP catheter, a variety of medical devices can be produced or used in accordance with embodiments of this invention, including without limitation, steerable catheters and guide wires, urologic or gynecologic catheters, coronary catheters (both venous and arterial), all in a wide variety of lengths and diameters. Embodiments including the shapeable shaft sections and the operating handle described herein may be used in a variety of medical and surgical procedures.

Furthermore, while the present invention has been described with reference to certain preferred embodiments or apparatuses, it is to be understood that the present invention is not limited to such specific embodiments or apparatuses. Rather, it is the inventor's contention that the invention be understood and construed in its broadest meaning as reflected by the following claims. Thus, these claims are to be understood as incorporating not only the preferred apparatuses described herein but all those other and further alterations and modifications as would be apparent to those of ordinary skilled in the art.

We claim:

1. A catheter comprising,
an elongated shaft having a proximal shaft section and a shapeable shaft section distal to the proximal section, an inner lumen extending within the proximal and shapeable shaft sections;
a shaping member slidably disposed within the inner lumen and extends into the shapeable shaft section;
a shape memory member adjacent to the shaping member in the inner lumen of the shapeable shaft section, wherein the shaping member and the shape memory member are coupled by a coupling coil; and
a handle having an operating lever, wherein the handle receives the proximal shaft section and the shaping member for operating the shapeable shaft section.

2. The catheter of claim 1 wherein a helical confining wire is disposed around the shaping member and the shape memory member to prevent the shaping member and the shape memory member from completely bowing apart when the shaping member is pulled or pushed.

3. The catheter of claim 2 wherein the helical confining wire defines a maximum distance between the shaping member and the shape memory member.

4. The catheter of claim 1 wherein a wire mesh is disposed around the shaping member and the shape memory member to prevent the shaping member and the shape memory member from completely bowing apart when the shaping member is pulled or pushed.

5. The catheter of claim 4 wherein the wire mesh defines a maximum distance between the shaping member and the shape memory member.

6. The catheter of claim 1 wherein the shapeable shaft section has a curvature and wherein the shape memory member provides a spring back force at the distal end of the shapeable shaft section that is opposite to the curvature of the shapeable shaft section.

7. The catheter of claim 1 wherein the shaping member has a flattened distal portion and wherein the flattened distal portion of the shaping member and the distal portion of the shape memory member each has a notched section and the coupling coil is wrapped around the notched sections of the elongated shaping member and the shape memory member.

8. The catheter of claim 1 further comprising a self-locking deflection system that comprises,
a rotor which is movably mounted within the handle, which has an engagement member secured thereto having a second friction engaging surface, which has at least one biasing element which urges the engagement member toward a first friction engaging surface so as to engage the second friction engaging surface with the first friction engaging surface; and
one of the first and the second friction engaging surfaces having increased frictional resistance to motion in a moving direction as the engagement member moves in the moving direction so as to hold the rotor in position upon release of pressure on the operating lever and to hold the elongated shaping member in a deflected position.

9. The catheter of claim 8 wherein the handle includes a rotatable wall on the exterior of the handle to which the operating lever is secured, and wherein the rotor is secured to an interior of the rotatable wall.

10. The catheter of claim 9 wherein the handle includes a flexible rack in the interior of the handle having a plurality of teeth and a flexible tail movably mounted within the handle.

11. The catheter of claim 10 wherein the handle includes a pin that passes through the rotatable wall, having one end secured to the operating lever and another end secured to the flexible tail of the flexible rack so that movement of the operating lever will cause movement of the flexible rack.

12. The catheter of claim 11 wherein the handle includes a slider secured to the proximal end of the shaping member and a gear assembly secured to the slider having a first gear engaging the flexible rack such that movement of the flexible rack will cause movement of the shaping member.

13. The catheter of claim 12 wherein the first gear of the gear assembly is mounted to a post rotatably secured to the slider.

14. The catheter of claim 13 wherein the gear assembly comprises a second gear mounted to the post rotatably secured to the slider and a second rack secured to the interior of the handle that is configured to receive teeth of the second gear.

15. The catheter of claim 1 wherein the catheter is an electrophysiology catheter having at least one electrode disposed on the exterior of the shapeable shaft section.

16. The catheter of claim 15 further comprising at least one temperature sensing element disposed adjacent to the at least one electrode.

17. The catheter of claim 1 further comprising at least one pressure sensor disposed on a distal tip of the shapeable shaft section.

18. The catheter of claim 1 is a linear ablation catheter.

19. The catheter of claim 1 is a tip ablation catheter.

20. The catheter of claim 1 further comprising an irrigation system, wherein the irrigation system provides liquid to the exterior of the shapeable shaft section to irrigate the at least one electrode.

21. An electrophysiology catheter comprising,
an elongated shaft having a proximal shaft section and a shapeable shaft section distal to the proximal section, an inner lumen extending within the proximal and shapeable shaft sections;
at least one electrode disposed on the exterior of the shapeable shaft section;
a shaping member slidably disposed within the inner lumen and extends into the shapeable shaft section;
a shape memory member adjacent to the shaping member in the inner lumen of the shapeable shaft section, wherein the shaping member and the shape memory member are coupled by a coupling coil, wherein the shaping member has a flattened distal portion and wherein the flattened distal portion of the shaping member and the distal portion of the shape memory member each has a notched section and the coupling coil is wrapped around the notched sections of the elongated shaping member and the shape memory member;
a handle having an operating lever, wherein the handle receives the proximal shaft section and the shaping member for operating the shapeable shaft section; and
a self-locking deflection system that comprises
a rotor which is movably mounted within the handle, which has an engagement member secured thereto having a second friction engaging surface, which has at least one biasing element which urges the engagement member toward a first friction engaging surface so as to engage the second friction engaging surface with the first friction engaging surface; and
one of the first and the second friction engaging surfaces having increased frictional resistance to motion in a moving direction as the engagement member moves in the moving direction so as to hold the rotor in position upon release of pressure on the operating lever and to hold the elongated shaping member in a deflected position;
wherein a helical confining wire is wrapped around the shaping member and the shape memory member to prevent the shaping member and the shape memory member from completely bowing apart when the shaping member is pulled or pushed and wherein the helical confining wire defines a maximum distance between the shaping member and the shape memory member.

22. The electrophysiology catheter of claim 21 wherein the handle includes a rotatable wall on the exterior of the handle to which an operating lever is secured, wherein the rotor is secured to an interior of the rotatable wall, wherein the handle includes a flexible rack in the interior of the handle having a plurality of teeth and a flexible tail movably mounted within the handle, wherein the handle includes a pin that passes through the rotatable wall, having one end secured to the operating lever and another end secured to the flexible tail of the flexible rack so that movement of the operating lever will cause movement of the flexible rack, wherein the handle includes a slider secured to the proximal end of the shaping member and a gear assembly secured to the slider having a first gear engaging the flexible rack such that movement of the flexible rack will cause movement of the shaping member, wherein the first gear of the gear assembly is mounted to a post rotatably secured to the slider, and wherein the gear assembly comprises a second gear mounted to the post rotatably secured to the slider and a rack secured to the interior of the handle that is configured to receive teeth of the second gear.

23. The catheter of claim 21 further comprising at least one temperature sensing element disposed adjacent to the at least one electrode, at least one pressure sensor disposed on a distal tip of the shapeable shaft section and an irrigation system, wherein the irrigation system provides liquid to the exterior of the shapeable shaft section to irrigate the at least one electrode.

24. An electrophysiology catheter comprising, an elongated shaft having a proximal shaft section and a shapeable shaft section distal to the proximal section, an inner lumen extending within the proximal and shapeable shaft sections;
at least one electrode disposed on the exterior of the shapeable shaft section;
at least one temperature sensing element disposed adjacent to the at least one electrode;
at least one pressure sensor disposed on a distal tip of the shapeable shaft section;
an irrigation system, wherein the irrigation system provides liquid to the exterior of the shapeable shaft section to irrigate the at least one electrode;
a shaping member slidably disposed within the inner lumen and extends into the shapeable shaft section;
a shape memory member adjacent to the shaping member in the inner lumen of the shapeable shaft section, wherein the shaping member and the shape memory member are coupled by a coupling coil, wherein the shaping member has a flattened distal portion, wherein the flattened distal portion of the shaping member and the distal portion of the shape memory member each has a notched section and the coupling coil is wrapped around the notched sections of the elongated shaping member and the shape memory member, wherein the shapeable shaft section has a curvature, and wherein the shape memory member provides a spring back force at the distal end of the shapeable shaft section that is opposite to the curvature of the shapeable shaft section;
a handle having an operating lever, wherein the handle receives the proximal shaft section and the shaping member for operating the shapeable shaft section; and
a self-locking deflection system that comprises
a rotor which is movably mounted within the handle, which has an engagement member secured thereto having a second friction engaging surface, which has at least one biasing element which urges the engagement member toward a first friction engaging surface so as to engage the second friction engaging surface with the first friction engaging surface; and
one of the first and the second friction engaging surfaces having increased frictional resistance to motion in a moving direction as the engagement member moves in the moving direction so as to hold the rotor in position upon release of pressure on the operating lever and to hold the elongated shaping member in a deflected position;
wherein a helical confining wire is wrapped around the shaping member and the shape memory member to prevent the shaping member and the shape memory member from completely bowing apart when the shaping member is pulled or pushed, wherein the helical confining wire defines a maximum distance between the shaping member and the shape memory member, wherein the handle includes a rotatable wall on the exterior of the handle to which an operating lever is secured, wherein the rotor is secured to an interior of the rotatable wall, wherein the handle includes a flexible rack in the interior of the handle having a plurality of teeth and a flexible tail movably mounted within the handle, wherein the handle includes a pin that passes through the rotatable wall, having one end secured to the operating lever and another end secured to the flexible tail of the flexible rack so that movement of the operating lever will cause movement of the flexible rack, wherein the handle includes a slider secured to the proximal end of the shaping member and a gear assembly secured to the slider having a first gear engaging the flexible rack such that movement of the flexible rack will cause movement of the shaping member, wherein the first gear of the gear assembly is mounted to a post rotatably secured to the slider, and wherein the gear assembly comprises a second gear mounted to the post rotatably secured to the slider and a rack secured to the interior of the handle that is configured to receive teeth of the second gear.
